(19) United States
(12) Patent Application Publication
(10) Pub. No.: US 2024/0169124 A1
(43) Pub. Date: May 23, 2024

(54) MULTIBODY SIMULATION

(71) Applicant: D. E. Shaw Research, LLC, New York, NY (US)

(72) Inventors: Brannon Batson, Brooklyn, NY (US); Brian Lee Greskamp, Minneapolis, MN (US); Bruce Edwards, New York, NY (US); Jeffrey Adam Butts, Beaverton, OR (US); Christopher Howard Fenton, Croton-On-Hudson, NY (US); Jeffrey Paul Grossman, Duluth, MN (US); Douglas John Ierardi, Valley Stream, NY (US); Adam Lerer, Cambridge, MA (US); Brian Patrick Towles, Chapel Hill, NC (US); Michael Edmund Bergdorf, New York, NY (US); Cristian Predescu, Lexington, MA (US); John K. Salmon, New York, NY (US); Andrew Garvin Taube, Brooklyn, NY (US)

(21) Appl. No.: 18/282,818

(22) PCT Filed: Mar. 18, 2022
(86) PCT No.: PCT/US2022/020915

§ 371 Date: Sep. 19, 2023

Related U.S. Application Data
(60) Provisional application No. 63/279,788, filed on Nov. 16, 2021.
(60) Provisional application No. 63/227,671, filed on Jul. 30, 2021.
(60) Provisional application No. 63/163,552, filed on Mar. 19, 2021.

(51) Int. Cl. G06F 30/25

(52) U.S. Cl. CPC G06F 30/25

(57) ABSTRACT

Improvements in a molecular-dynamic simulator provide ways to save energy during computation and reduce die area consumed on an integrated circuit. Examples of such improvements include different interaction modules for different ranges, the use of streaming along rows while multicasting along columns in an array of interaction modules, the selection of computation units based on balancing computational costs and communication costs, the use of fences in networks that connect computation units, and the use of bond calculators to carry out specialized bond calculations.

### RELATED APPLICATIONS

This application claims the benefit of U.S. Provisional Applications Nos. 63/163,552 filed 19 Mar. 2021, 63/227,671 filed 30 Jul. 2021, and 63/279,788 filed 16 Nov. 2021, the contents of which are incorporated herein by reference.

### STATEMENT OF PRIOR DISCLOSURE BY THE INVENTOR(S)

“Anton 3: twenty microseconds of molecular dynamics simulation before lunch,” by Shaw, David E., Peter J. Adams, Asaph Azaria, Joseph A. Bank, Brannon Batson, Alistair Bell, Michael Bergdorf et al., In Proceedings of the International Conference for High Performance Computing, Networking, Storage and Analysis, pp. 1-11. Nov. 2021,

### DOI:10.1145/3458817.3487397.

“The Specialized High-Performance Network on Anton 3,” by Keun Sup Shim, Brian Greskamp, Brian Towles, Bruce Edwards, J. P. Grossman, David E. Shaw, arXiv:2201.08357v1, Jan-2022.

These publications are incorporated herein by reference.

### BACKGROUND OF THE INVENTION

This invention relates to multibody simulation, and more particularly to a circuit implementation of an apparatus for simulation of molecular dynamics.

### SUMMARY OF THE INVENTION

A number of examples of circuit implementations of and operating procedures for an apparatus for multibody simulation are described in the following U.S. Patents, which are incorporated herein by reference: U.S. Pat. No. 7,707,016, entitled “ORTHOGONAL METHOD,” U.S. Pat. No. 7,526,415, entitled “GRID BASED COMPUTATION FOR MULTIPLE BODY SIMULATION,” and U.S. Pat. No. 8,126,956, entitled “APPROACHES AND ARCHITECTURES FOR COMPUTATION OF PARTICLE INTERACTIONS.”

This document describes a number of aspects that can be used in conjunction with the previously described approaches, for example, substituting implementations of subsystems or modifying subsystems with aspect presented herein.

In a number of implementations, an apparatus for multibody simulation simulates a physical volume that includes a number of particles. In the context of simulation of molecular dynamics, the particles include atoms, groups of which may form molecules.

The apparatus includes a number of interconnect processing nodes, which may be arranged in a three-dimensional array. In a number of uses of the apparatus, there exists a one-to-one association between processing nodes and physical regions of the physical volume being simulated. Embodiments include those in which the physical regions are cubes, those in which they are rectangular prisms, and those in which they are arranged in with the same neighboring relationships as the processing nodes. In at least some implementations, processing nodes have communication paths to their direct neighbors. These paths form a toroid.

As described in the prior patents, data for a particular particle is stored in a processing node associated with the physical location of that particle. Computation of particle interactions generally involves exchanging information about particles so that processing nodes can compute pairwise interactions, and for at least some particles exchanging force information so that processing nodes can update the locations (and velocities) of those particles.

A number of new features set forth below, which can be used alone or in combination with each other, provide technical improvements to a practical problem of accurately simulating a physical system in a circuit-based system.

One improvement is that the total amount of energy consumed for a given simulation is reduced. Such a reduction in energy enables implementation of faster and/or smaller systems.

Another improvement is that the time needed to simulate a physical system is reduced, not merely by virtue of using a faster circuitry or general-purpose processors, but by the specific arrangement of computation and inter-node communication that may make better use of available circuitry, for example, by introducing particular combinations of processing elements, arranging communication and computation aspects to reduce latency and thereby reduce the time needed for each simulation cycle, and making more efficient use of communication links between processors.

All implementations and methods described herein are non-abstract and provide a technical effect. As used herein, Applicant, acting as his own lexicographer, hereby defines “non-abstract” to mean the opposite of “abstract” as that term has been defined by the Federal Circuit and Supreme Court as of the filing date of this application. As a result, any person who construes the claims as abstract will be construing the claims in a manner directly contrary to the specification.

In one aspect, the invention features a hybrid method for interacting two atoms in a pair of atoms. According to this method, a set of one or more computation nodes is used to interact a pair of atoms. The set is selected by balancing the cost of having to communicate data concerning atoms between communication nodes within the set and the computational complexity associated with computing the interaction.

As used herein, the verb “to interact” shall mean to carry out the computations required to estimate a change in state (e.g. position, momentum, charge, etc.) of the two atoms that results from an interaction between the two atoms. Throughout this disclosure, the terms “atom” and “particle” shall be used interchangeably.

As used herein, the term “atom” is not intended to necessarily mean a nucleus with its retinue of electrons. In the context of molecular dynamics, an “atom” is used in its original sense as that which is treated as an indivisible unit during a simulation. As such, an “atom” could be a nucleus, a nucleus and one or more electrons, plural nuclei bonded together, e.g., a molecule, or a functional group that is part of a much larger molecule.

In a molecular-dynamics simulator, interacting two atoms requires information about the two atoms. This information must be available at whichever computation node will carry out the interaction. A particular computation node has information about some but not all atoms. If the node already has the information associated with both atoms of a pair, there is no communication cost associated with transmitting such information. On the other hand, if the node does not have information about one of the atoms, then a communication cost is incurred as a result. In some cases, the node has no information on either atom. This incurs an even larger communication cost.

The implementation described herein chooses between a first method, which has higher communication costs and lower computational complexity, and a second method, which has lower communication costs and higher computational complexity. In the present case, the first method is the Manhattan Method, and the second method is the Full Shell method. For each interaction, the simulator weighs the added communication cost of the first method against the higher computation cost of the second method and selects the set of computation nodes that gives the better performance for each interaction.

When compared with existing Neutral Territory methods, such as that described in U.S. Pat. No. 7,707,016, the Manhattan Method often improves performance as a result of having a smaller import volume among nodes and better computational balance across nodes. The Manhattan Method computes the interaction on the one of the nodes that contains the particle that is furthest away from an internode boundary, in a physical space. It then returns the shared result to another node.

The Full Shell method is significantly more computationally complex than either of the foregoing methods. However, it also requires much less communication. This savings in communication arises because interactions are computed at both atoms' home nodes and therefore are not returned back to a paired node.

In another aspect, the apparatus includes circuitry at the processing nodes for evaluating pairwise interactions between particles.

The computation of the interaction between a pair of particles may have different requirements depending on the separation of the particles. For example, particles that are farther away from one another may require less computation because the interaction is less complex than if they were closer to one another. The magnitude of characteristics of the interaction may be smaller. The computed characteristic of the interaction may be smaller.

To accommodate this, it is useful to have multiple types of processing elements for computing pairwise interactions, with the type of processing element being selected according to the separation of the particles.

As an example, in simulation of molecular dynamics, non-bonded particles have more complex behavior near each other than when further away. Near and far are defined by cutoff radii of spheres around point particles. Due to near uniform density of particles distributed in a liquid and the cutoff ranges, there are typically thrice as many particles in the far region as there are in the near region. The apparatus exploits this by steering pairs of particles that are close to each other to a big interaction module that is capable of carrying out more complex processing. Conversely, pairs of particles that are far from each other are steered to a small interaction module that carries out lower precision calculations and ignores certain phenomena that are of significance only when particles are close enough to each other.

The use of “big” and “small” is apt because the big interaction module is physically bigger in size. It consumes more chip area than the small interaction module and also consumes more energy per interaction. A processing node may have a greater number of the “small” processing elements that “big” processing elements to accommodate spatial distribution of particles in the simulation volume.

A portion of the total area of each integrated circuit holds interaction circuitry that forms a computation pipeline. This interaction circuitry carries out the foregoing interactions.

Unlike a general-purpose computer, the computation pipeline is a minimally-configurable hardware module with only limited functionally. However, what it does, it does well. The interaction circuitry consumes far less energy to carry out an interaction than a general-purpose computer would consume for the same interaction. This interaction circuitry, which can be regarded as a pairwise particle interaction module, is the true workhorse of the integrated circuit.

Other portions of the substrate have, formed thereon, logic circuitry. Such logic circuitry typically comprises transistors that are interconnected to transform electrical voltages into an output voltage. The result of such transformation is that of sending or receiving information represented by voltages towards the interaction circuitry, providing temporary storage of information, or otherwise conditioning the information.

In another aspect, in general, given data for two sets of particles, a processing node determines, according to a distance between the particles, (1) whether to evaluate the interaction between the particles, and/or (2) which processing element should be used to compute the interaction between the particles.

Some examples use a strict threshold on distance between particles in determining whether to evaluate the interaction. This helps avoid, for example, inadvertently “double counting” the interaction (e.g., the forces on the particles).

In other examples, the distance between the particles determines which of different types of processing elements of the node to use for the interaction. This is particularly advantageous since different processing elements carry out calculations of different levels of accuracy. This makes it possible to choose which level of accuracy is most appropriate for a particular interaction.

In this aspect, the distance-based decisions (i.e., (1) and (2) above) are performed in two stages with increasing precision and/or increasing computational cost.

For example, in the first stage, pairs of particles are excluded if they are guaranteed to exceed a threshold separation. As another example, in a second stage, pairs of particles not excluded by the first stage are processed according to their separation, for example, to further exclude pairs of particles that exceed the threshold separation and/or to select a processing element according to the separation. For example, the second stage makes a three-way determination for a particle pair: is one particle is within a near region of the second particle (e.g., in which case the pair are evaluated using a “big” processing element), if one particle is within a far region of the second particle (e.g., in which case the pair are evaluated using a “small” processing element), or if one particle is outside the far region's cutoff radius of the second particle (e.g., in which case the interaction of the pair is not further evaluated).

Interaction between atoms includes taking into account phenomena whose significance varies with distance between the atoms. In recognition of this, it is useful to define a threshold distance from an atom. If an interatomic distance between first and second atoms of a pair of atoms exceeds this threshold, a first interaction module will be used; otherwise, a second interaction module will be used. The two interaction modules differ in complexity, with the first interaction modules ignoring at least one phenomenon that is taken into account in the second interaction modules. For example, when the distance is small, quantum mechanical effects are significant enough to take into account. Such effects can be ignored when the distance is large.

The first interaction module is physically larger than the second and thus takes up more die area. Additionally, the first interaction consumes more energy per interaction than the second interaction module.

In general, there exists a sphere centered on the first atom. Atoms that lie beyond the sphere are not interacted at all. Atoms that lie within the sphere but beyond a threshold radius are interacted using the second interaction module. All other atoms are interacted in the first interaction module.

To steer interactions to the correct interaction module, it is useful to have matching circuitry that determines interatomic distance and either discards the proposed interaction or steers the interaction to the first interaction module or second interaction module accordingly based whether the interatomic distance is below or above the threshold radius.

For uniform density of atoms, it is expected that more atoms will lie in the portion of the sphere outside the threshold radius. As a result, it is useful to have two or more second interaction modules. This promotes further parallelism for interactions of the second type.

In some embodiments, atoms are first saved in memory and then streamed into the interaction circuitry, and in particular, to matching circuitry that steers atoms to appropriate interaction modules. The matching circuitry implements a two-stage filter in which a low-precision stage is a coarse and inclusive filter. In each clock cycle, the low-precision stage computes interatomic distances between each streamed atom and a number of stored atoms that are to potentially be interacted with streamed atoms.

It is useful for each atom to have a “type.” Knowing an atom's “type” is useful for selecting a suitable interaction method to be used when that atom is a participant in the interaction. For example, when the types of two atoms are known, it is possible to consult a look-up table to obtain information concerning the nature of the pairwise interaction between those two atoms.

To avoid the unwieldiness associated with large tables, it is useful for the interaction module to have a two-stage table in which a first stage has interaction indices, and a second stage has the relevant interaction types associated with each interaction index. The interaction index represents a smaller amount of data than the information concerning the atom's type. As such, the first stage of the table, which must physically exist on a die, consumes a smaller area of the die. Accordingly, it also consumes less energy to maintain that information.

As noted above, the interaction circuitry that forms the computation pipeline has only limited functionality. For some interactions, it is necessary to carry out operations that the interaction circuitry is unable to carry out. For such cases, an interaction type associated with one of the participating atoms indicates that a special operation is needed. To carry this out, the interaction circuitry implements a trap-door to an adjacent general-purpose core, referred to herein as a “geometry core.” The geometry core is generally less energy efficient than the interaction circuitry. However, it can carry out more complex processing. This implementation thus retains the energy efficiency associated with the interaction circuitry while having the ability to occasionally subcontract a portion of a calculation to a less efficient geometry core.

As introduced above, communication between processing nodes involves exchanging information about the states of particles. Such information includes one or more of position, velocity, and/or forces on particles. In successive iterations of a simulation, a particular pair of processing nodes may send information about a same particle.

In another aspect, in general, a reduction in communication requirements is achieved by referencing previously communicated information. For example, a receiving node may cache information (e.g., a mass of a particle), and a transmitting node may in subsequent iterations send a reference (e.g., a tag) to the cached data rather than resending the full data.

In another aspect, in general, a transmitting node and a receiving node share information from previous iterations that is used to predict the information to be transmitted in a current information. To transmitting node then encodes the information to be transmitted in the current iteration relative to the shared prediction, thereby reducing the amount of data to be transmitted. For example, to the extent that a transmitting node and a receiving node share a previous position and velocity of a particle, each can predict a current position and velocity, for example, by moving the particle at that previous velocity and assuming the velocity remains constant. Therefore, the transmitting node only has to send a difference between the current position and the predicted position and/or the difference between the current velocity and the predicted velocity. Similarly, forces may be predicted in a like manner, and differences between predicted and computed forces may be sent.

In another aspect, a communication infrastructure (e.g., inter-node communication circuitry) connecting processing nodes in the system includes circuitry for synchronization of communication between nodes. Among the embodiments of the foregoing infrastructure are those in which a node emits a “fence” message that indicates that all of a set of messages have been sent and/or that indicates that message sent from that node after the fence message must be delivered to destinations after the fence message.

Also among the embodiments of the foregoing infrastructures are those in which the communication infrastructure determines when to send a message to a destination node that is indicative of all messages from a set of source nodes as having been delivered. Among these are embodiments in which the communication infrastructure processes fence messages from the set of source nodes and delivers a fence message to a destination node when all the fence messages from the source nodes have been received. Such infrastructure-based processing of fence messages can avoid a need for having to send “N 2” messages between pairs of processing nodes.

In another aspect, a processor synchronization mechanism for a large multiprocessor computer connected by a network makes use of fences. A fence is a barrier that guarantees to a destination processor that no more data will arrive from all possible sources. In some embodiments, fences are global barriers, i.e., synchronizing all processors in the computer. In other embodiments, fences are selective barriers that synchronize regions of the computer.

Among the embodiments are those in which each source sends a packet to each destination indicating the last data was sent and each destination waits until packets from each source have been received. In a computer with N processors, a global barrier would need O(N²) packets to traverse the network from all source to destination processors. An alternative fence mechanism requires only O(N) packets be sent and received by end-point processors. Further embodiments include a network using multicast and counters to reduce fence network traffic and processing at endpoints, thereby reducing power consumption and reducing the physical area used on a silicon chip, thereby reducing the cost of manufacture.

In another aspect, the invention includes interaction modules for computing interactions between pairs of atoms in which computational units, referred to herein as “tiles,” form a two-dimensional array of rows and columns within an integrated circuit, or “chip.” A given tile transmits and receives information concerning particles either with an adjacent tile in the same column or an adjacent tile in the same row. For ease of exposition, information concerning a particle shall be referred to simply as “the particle.”

A tile stores a set of particles, hereafter referred to as “stored-set particles.” During the course of simulation, that tile receives a stream of particles, hereafter referred to as “stream-set particles.” In the course of the simulation, the tile interacts each stream-set particle with each stored-set particle. At each time step in the simulation, the stream-set particles that have been interacted by the tile move along that tile's row to a subsequent tile, to be interacted with stored-set particles at that subsequent tile. Meanwhile, the tile receives new stream-set particles from a preceding tile in that tile's row.

To carry out this row-wise streaming, there exists a dedicated streaming network. This dedicated streaming network features position buses and force buses. The position buses obtains information concerning a particle's position from memories at the chip's edge and streams it through the interaction circuitry from one tile to the next. For each particle, the force buses accumulate forces acting on that particle as those forces are computed by the interaction modules through which that particle passes.

As noted above, a tile is also able to communicate with other tiles in its column. This communication does not involve the streamed-set particles. It involves the stored-set particles. In particular, stored-set particles at a tile are multicast to tiles in that tile's column. As a result, stored-set particles are replicated across all tiles in the same column. This makes it possible to interact the stored-set particles with different streamed-set particles at the same time.

A difficulty that arises is that forces acting on a stored-set particle as a result of interactions with streamed-set particles in one row will not necessarily be available to the corresponding stored-set particle in another row. To address this difficulty, the forces that are computed for streamed-set particles in a row are reduced in-network upon unloading by simply following the inverse of the multicast pattern that was used to multicast the stored-set particles in the first place.

Additionally, no tile is permitted to start unloading stored-set particles until all tiles in the same column are ready to start unloading. To implement this, it is useful to provide a column synchronizer in the form of a four-wire synchronization bus across all tiles within a column. Such a synchronization bus avoids network deadlock and provides low-latency synchronization.

In another aspect, the invention includes a bond calculator that acts as a coprocessor to assist a general-purpose processor in carrying out certain specialized calculations that concern particular types of bonds between atoms, and in particular, covalent bonds. The general-purpose processor launches such a calculation by providing information concerning the atoms and the nature of the bond to the bond calculator and retrieving a result of such processing from the bond calculator's output memory.

Embodiments of bond calculators support one or more of responses of bonds to forces. Such responses include a change in bond length, such as an extension or contraction of the bond, a change in the bond angle, which can arise when three atoms are bonded, and a change in the bond's dihedral or torsion angle, such as that which can arise when four bonded atoms are present.

These responses to forces are particularly common in molecular simulation. As a result, it is particularly useful to offload processing associated with determining these responses to compact and specialized circuitry. Doing so reduces energy needed to calculate such state changes in the atoms.

In some embodiments, interactions between particles take the form of a difference of exponentials, for example, of the form exp(-ax)-exp(-bx), or as the evaluation of an integral representing a convolution of electron cloud distributions. While it may be possible to compute the two exponentials separately and then take the difference, such differences may be numerically inaccurate (e.g., differences of very large numbers). A preferable approach is to form one series representation of this difference. For example, the series may be a Taylor series or a Gauss-Jacobi quadrature-based series. Furthermore, the number of terms needed to maintain precision of the overall simulation will in general depend on the values of ax and bx. Therefore, in computing the pairwise terms, for example, in the Particle-Particle-Interaction circuitry (PPIM), different particular pairs of particles, or different criteria based on the difference (e.g., absolute difference, ratio, etc.) in the values of ax and bx, can determine how many series terms to retain. By reducing the number of terms (e.g., to a single term for may pairs of particles), for example, when the two values are close, the overall computation of all the pairwise interactions may be reduced substantially while maintaining overall precision, thereby providing a controllable tradeoff between accuracy and performance (computation speed and/or hardware requirements).

In some implementations, the same values (e.g., forces on particles) are computed redundantly in different processors, for example, to avoid communication cost. For example, such redundant computation may occur in the “Full Shell” method. There are also situations in which systematically truncating or rounding results may be detrimental to the overall simulation, for example, by introducing bias over a series of iterations. For example, repeatedly rounding down may make an integration over time significantly too small.

One approach to avoiding accumulated bias resulting from rounding is successive time steps is to add a small zero-mean random number before rounding or truncating a value computed for a set of particles. Such an approach may be referred to as dithering. However, when performing redundant computations in different processors, there is no reason that pseudo-random numbers generated at the different processors will be the same, for example, because of difference in the order of random number generation. With different random numbers, the rounded or truncated values may differ, that the simulation may not stay in total synchronization across processors.

A preferred approach is to use data-dependent random number generation, where exactly the same data is used at all nodes that compute a value for a set of particles. One way to generate a random value is to use coordinate differences between the particles involved in the computation as a random seed for generating the random value(s) to be added before rounding or truncation. In some embodiments, the low order bits of the absolute differences in each of the three geometric coordinate directions are retained and combined as an input to a hash function whose output is used as the random value or that is used as a random seed of a pseudo-random number generator that generates one or more random numbers. When there are multiple computations involving the set of particles, the same hash is used to generate different random numbers to add to the results of computations. For example, one random number if split into parts, or a random number generator is used to generate a sequence of random numbers from the same seed. Because the values of the coordinate distances are exactly the same at all the processors, the hash value will be the same, and therefore the random numbers will be the same. Distances between particles may be preferable to absolute locations because the distances are invariant to translation and toroidal wrapping while absolute locations may not be. Computing differences in coordinate directions does not incur rounding error and therefore may be preferable to Euclidean (scalar) distances.

Embodiments, examples, and/or implementations make use of various combinations of the approaches describe above, and advantages of individual approaches, including reduction in communication requirements measured in number of bits of information transmitted, reduction in latency of communication, measured in absolute time or relative to time required to perform certain computations, reduction in absolute (i.e., “wall-clock” time) to perform a given simulation over a simulated time and for a number of simulated time steps, reduction in the number of computational operations required to perform a simulation, distribution of computations to particular computational modules to reduce computation time and/or power and/or circuit area required, and/or synchronization between distributed modules using fewer communication resources and/or providing more synchronized operation using network communication primitives, may be achieved without requiring their use in combination with other of the approaches. Yet other advantages are evident from the description below.

### DESCRIPTION OF DRAWINGS

FIG. 1 is a logical block diagram of a computation system comprising computation nodes arranged in a three-dimensional grid.

FIG. 2 is an illustration of a structure of an application specific integrated circuit of a computational node of FIG. 1.

FIG. 3 is a logical block diagram of a core tile of the circuit of FIG. 2.

FIG. 4 is a logical block diagram of an edge tile of the circuit of FIG. 2.

FIGS. 5A-C are diagrams representing three different examples of communication among computation nodes when computing interactions between atoms.

FIG. 6 is a logical block diagram of a pairwise particle interaction module core tile of FIG. 3.

### DETAILED DESCRIPTION

1 Overview

1.1 Hardware Architecture

The description below discloses a hardware system as well as computational and communication procedures that are executed on that hardware system to implement a molecular dynamics (MD) simulation. This simulation predicts the three-dimensional movements of atoms in a chemical system over a large number of discrete time steps. During each time step, inter-atomic forces among the atoms are computed using physics-based models. These inter-atomic forces consist of bond terms that model forces between small groups of atoms usually separated by 1-3 covalent bonds, and non-bonded forces between all remaining pairs of atoms. At each time step the forces on a given atom are summed to give a total force on the atom, which (by Newton's second law) directly determines the acceleration of the atom and thus (by integrating over time) can be used to update the atomic positions and velocities of the atoms to their values to be used in the next time step. Without approximating some calculations, the number of inter-atomic forces computed on each time step scales quadratically with the number of atoms, meaning there is an enormous increase in time-to-solution with increasing system size. Furthermore, time steps on the order of a femtosecond are required for stable and accurate integration; around a billion time steps are thus necessary to simulate a microsecond of atomic motions.

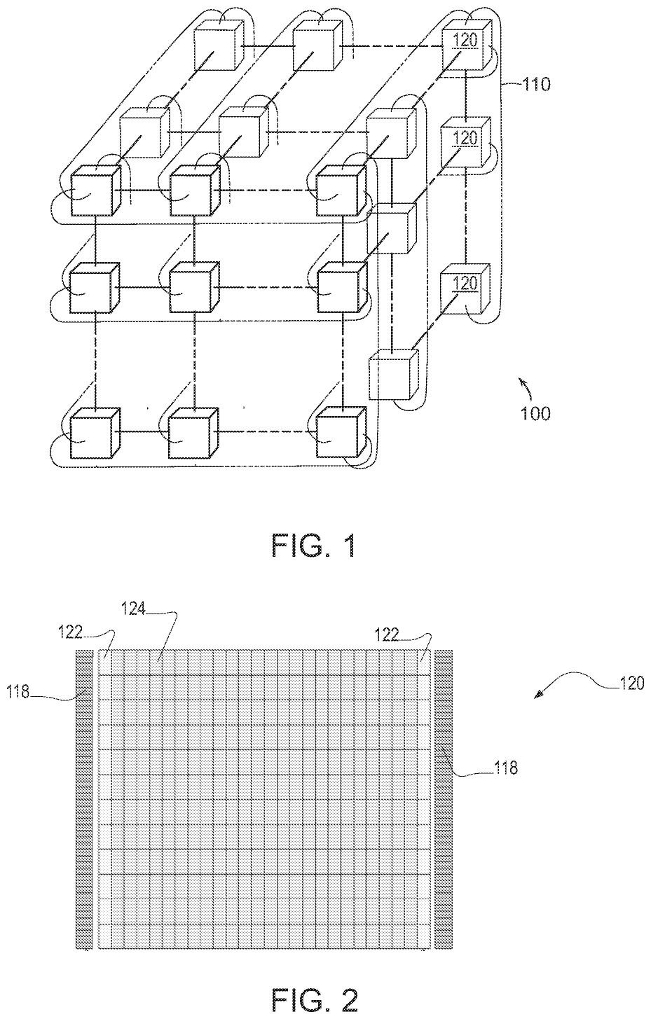

Referring to FIG. 1, the computation system 100 includes a three-dimensional arrangement of computational nodes 120 implemented as separate hardware elements. For example, 512 nodes are arranged in an 8×8×8 array, recognizing that different numbers of nodes may be used. In general, the nodes 120 host both computation and communication functions that are implemented in application-specific hardware and/or software that is executed on special-purpose or relatively general-purpose processors integrated in the nodes. The nodes 120 are linked internode communication network that provides communication capabilities lining the nodes. In the embodiment illustrated in FIG. 1, the internode communication network includes a number of node-to-node communication links 110 that couple adjacent nodes in a toroidal arrangement in the three dimensions of the node array. That is, as shown in FIG. 1, each node 120 is coupled to six links, two links in each of the three dimensions (e.g., x, y, and z). Although the nodes are illustrated in FIG. 1 as cubes, with links being coupled to each of the six faces of each cube, other physical arrangements of the nodes (e.g., in electronics racks) are used.

Each node includes communication elements, including routers that support communication between nodes that are not adjacent. As discussed further below, such routers are referred to as “edge routers.” Furthermore, each link 110 is generally made up of multiple bidirectional channels, that are in turn composed of one or more serial “lanes.” For example, each link 110 may consist of 16 lanes, such that a node has an aggregate of 6×16=96 lanes connected to other nodes 120 of the system. Therefore, the edge routers provide communication paths that pass communication between different lanes coupled to a node.

Referring to FIG. 2, each node 120 includes an application specific integrated circuit (ASIC) that is laid out as a two-dimensional array of cores (also referred to as “tiles”) that includes a central array of core tiles 124, and on two opposing boundary sides of that array, linear arrays of edge tiles 122. For example, the central array includes 12×24 core tiles, while each array of edge tiles 122 has 12 tiles. That is there are a total of 24 edge tiles. Each edge tile 122 is coupled to a number of serial channels, for example, with each edge tile being coupled to 4 serial channels via respective serializer-deserializer modules (SERDES) 118. Generally, the edge tiles 122 provide communication services for inter-node communication as well as between the inter-node communication network and one or more internal networks within the node, while the core tiles 124 provide computation services for the simulation, as well supporting communication on the internal networks on the node.

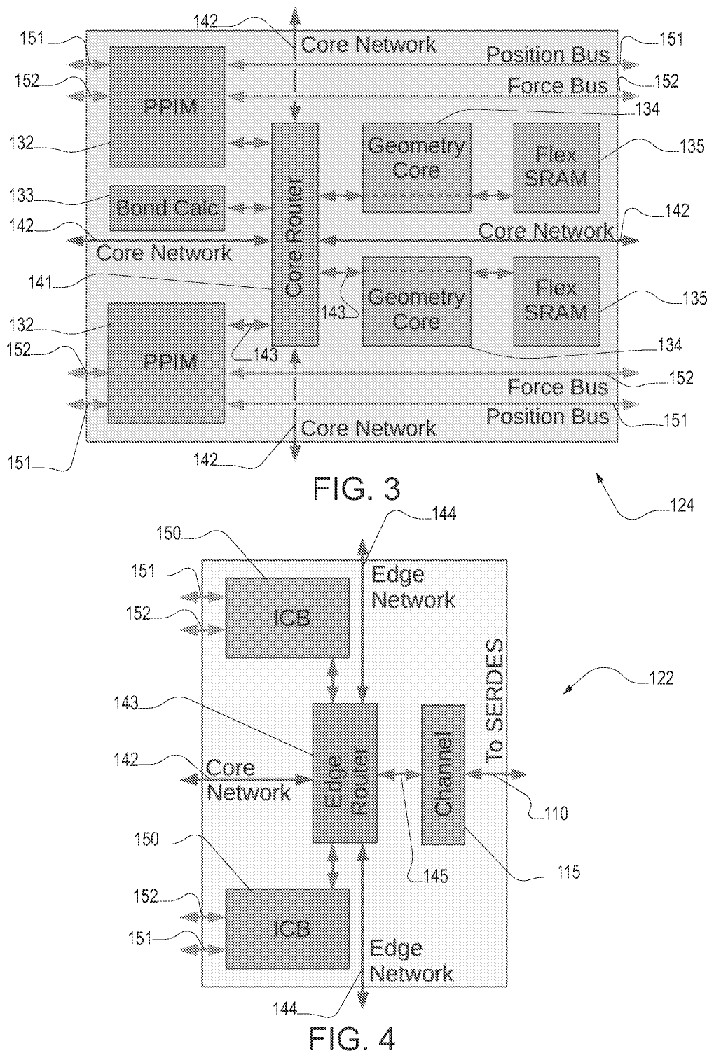

FIG. 3 shows the components of a core tile 124 in more detail. A network router (also referred to as a “core router”) 141 connects computational blocks in the tile to a general-purpose 2D mesh network-on-chip, which includes links 142 coupling adjoining core tiles 124. In addition to the mesh network, dedicated buses are used to distribute data inputs and outputs for simulation computations. These buses include a position bus 151 and a force bus 152. As will be described in detail below, a significant part of the computation relates to determining the forces exerted between pairs of atoms, and that computation is hosted in two pairwise point interaction modules (PPIMs) 132 on each tile, with these PPIMs receiving position information over the position bus 151 and providing force information over the force bus 152. In addition, the PPIMs serve to pass along data between the PPIMs to enable each PPIM to communicate with an edge tile 122. Each core tile 124 also includes a further computation module, referred to as the bond calculator (BC) 133, that handles computation of forces related on bonded atom. Finally, two relatively more general processing modules handle all remaining computation at each time step that is not already handled by the BC 133 or PPIMs 132. These modules are referred to as the geometry cores (GCs) 134, and their associated memories 135 (denoted “flex SRAM” in FIG. 3).

Referring to FIG. 4, each edge tile 122 contains the logic for the off-chip links 110 (channels), with each channel connecting to one of the chip's six neighbors in the 3D torus using a group of SERDES 118. Each channel also connects to an edge router 143, which forms an edge network with the other edge tiles via links 144 on the same edge of the node 120 (i.e., along the array of 12 edge tiles 122 at each end of the node as illustrated in FIG. 2), allowing traffic to “turn” across dimensions in the inter-node network. The edge router 143 also connects to the core tile's 2D mesh network via a link 142 for injection and ejection of data and to a channel adapter 115, which connects via the SERDES 118 to the inter-node links 110. Finally, interaction control blocks (ICBs) 150 connect the edge router to the force bus 152 and position bus 151, which run across the array of core tiles 124 as described above. The ICBs 150 include large buffers and programmable direct memory access (DMA) engines, which are used to send atom positions onto the position buses 151. They also receive atom forces from the force buses 152 and send them to the edge network for delivery to the Flex SRAMs 135.

Routing of communication packets on the 2D mesh network at each node uses a dimension-order routing policy implemented by the core routers 141. Routing in the 3D torus network makes use of a randomized dimension order (i.e., one of six different dimension orders). For example, the order is randomly selected for each endpoint pair of nodes.

The system 100 is, in general, coupled to one or more other computational systems. For example, initialization data and/or software is provided to the system 100 prior to the simulation and resulting position data is provided from the system 100 during the simulation or after completion of the simulation. Approaches to avoiding deadlock include using a specific dimension order for all response packets, and using virtual circuits (VCs).

1.2 Computational Architecture

The molecular dynamics simulation determines the movement of atoms in a three-dimensional simulation volume, for example, a rectilinear volume that is spatially periodically repeating to avoid issues of boundary conditions. The entire simulation volume is divided into contiguous (i.e., non-overlapping) three-dimensional boxes, which generally have uniform dimensions. Each of these boxes is referred to as a “homebox.” Each homebox is associated with one of the nodes 120 of the system (which may be referred to as the “homebox's node”, most typically in a one-to-one relationship such that the geometric relationship of nodes is the same as the geometric relationship of homeboxes (and therefore in the one-to-one case the homebox may be referred to as the “node's homebox”). In the one-to-one relationship case, adjacent homeboxes are associated with adjacent nodes. Note that in alternative embodiments each node may host multiple homeboxes, for example, with different parts of each node being assigned to different homeboxes (e.g., using different subsets of tiles for each homebox). The description below assumes a one-to-one association of nodes and homeboxes for clearer exposition.

At any point in simulated time, each atom in the simulation volume resides in one of the homeboxes (i.e., the location of the atom is within the volume of the homebox). At least that homebox's node stores and is responsible for maintaining the position and velocity information for that atom. To the extent that any other nodes have and rely on position and velocity information for that atom, the information is guaranteed to be identical (e.g., bit exact) to the information at the atom's homebox node. The simulation proceeds in a series of time steps, for example, with each time step representing on the order of a femtosecond of real time.

During each simulated time step, inter-atomic forces among the atoms are computed using physics-based models. These inter-atomic forces consist of bond terms that model forces between small groups of atoms usually separated by 1-3 covalent bonds, and non-bonded forces between all remaining pairs of atoms. The forces on a given atom are summed to give a total force on the atom, which (by Newton's second law) directly determines the acceleration of the atom and thus (by integrating over time) can be used to update the atomic positions and velocities to their values at the next time step. Without approximating some calculations, the number of inter-atomic forces computed on each time step scales quadratically with the number of atoms, meaning there is an enormous increase in time-to-solution with increasing system size. Furthermore, time steps on the order of a femtosecond are required for stable and accurate integration; around a billion time steps are thus necessary to simulate a microsecond of atomic motions.

To make such simulations computationally tractable, the forces among non-bonded atoms are expressed as a sum of range-limited forces and long-range forces. Range-limited forces decay rapidly with distance and are individually computed between pairs of atoms up to a cutoff distance. Long-range forces, which decay more slowly with distance, are computed using a range-limited pairwise interaction of the atoms with a regular lattice of grid points, followed by an on-grid convolution, followed by a second range-limited pairwise interaction of the atoms with the grid points. Further description of the approach to computation of the long-range forces may be found in U.S. Pat. No. 7,526,415, as well in Shan, Yibing, John L. Klepeis, Michael P. Eastwood, Ron O. Dror, and David E. Shaw, “Gaussian split Ewald: A fast Ewald mesh method for molecular simulation.” The Journal of Chemical Physics 122, no. 5 (2005): 054101.

The force summation for each atom is implemented as a distributed hardware reduction, with, in general, terms of a summation to determine the total force on any particular atom being computed at multiple different nodes and/or terms being computed at different tiles at one node and/or in different modules (e.g., PPIMs in one tile). At each node, different types of forces (e.g., bonded, range-limited, and long-range) are, in general, computed in different types of hardware modules at a node. Parallelism is achieved by performing force computations at different nodes 120 and at different modules (e.g., in different core tile 124 and/or different modules within a tile) within each node. As discussed further below, computation versus communication tradeoffs are chosen to reduce the overall simulation time (i.e., the actual computation time for a fixed simulated time) by pipeline computation (e.g., “streaming”), communicating required information for a particular force computation to one node and distributing the result in return to reduce total computation, and/or using redundant computation of the same forces at multiple nodes to reduce latency of returning the results.

Each time step generally involves overlapping communication and computation distributed among the nodes 120 and communication links 110 of the system. In at least some embodiments, at the start of a time step, at least some computation may begin at the computation nodes, for example, based on interactions between pairs of atoms where both atoms of the pair are located in the same homebox and therefore at the same node. Also beginning at the start of the time step, information about atoms (e.g., the positions of the atoms) is communicated from (i.e., “exported” from) nodes where that information is stored (or otherwise known) to nearby nodes (e.g., to nodes that may have atoms within the cutoff radius of an exported atom). As the information for atoms arrives at the other nodes (referred to as being “imported” by/to those nodes), further computation may begin to determine the interactions (e.g., the force terms) between atoms in different homeboxes. As the computations between atoms in whose positions are different homeboxes are computed, the results (e.g., force terms) may be sent back to the nodes from which the atom information was imported. Note that computation may be overlapped with communication, whereby importing of position information may be taking place at a node at the same time as computation of interactions with imported atoms at the same time as exporting force information of atoms that were previously imported. In parallel to computation of bonded and range-limited forces, long-range forces are computed using, for example, grid-based approaches addressed above. For each atom, once all the force terms on that atom are known at a node (e.g., at the node in whose homebox the atom was located at the start of the time step), the total force is computed for that atom and its position may be updated. When all the positions of the atoms in the overall system have been updated, the time step can finish and then the next time step can begin.

Approximations are also optionally used to reduce computational demand in at least some embodiments. For example, certain types of forces are updated less frequently than others, for example, with long-range forces being computed on only every second or third simulated time step. In addition, rigid constraints are optionally used to eliminate the fastest motions of hydrogen atoms, thereby allowing time steps of up to ˜2.5 femtoseconds. Optionally, the masses of hydrogen atoms are artificially increased allowing time steps to be as long as 4-5 fs.

About 10⁴ numerical operations are required per atom for each time step, which translates to around 10¹⁸ operations per microsecond of simulated time on a system of one million atoms, even combining these optimizations and approximations. This computational intensity is addressed in part by use of one or more of the techniques described below, recognizing that unless otherwise set forth, no one of the techniques is essential to the operation of the system.

2 Pairwise Computations

As introduced above, one part of the computation procedure relates to computing the effects of non-bonded interactions between pairs of atoms that are within a cutoff radius of each other (i.e., range-limited interactions). For any one atom, this computation involves summing forces (i.e., direction and magnitude and/or vector representations) exerted on it by the other atoms that are within the cutoff radius of it to determine the total (aggregate) force of all these non-bonded interactions.

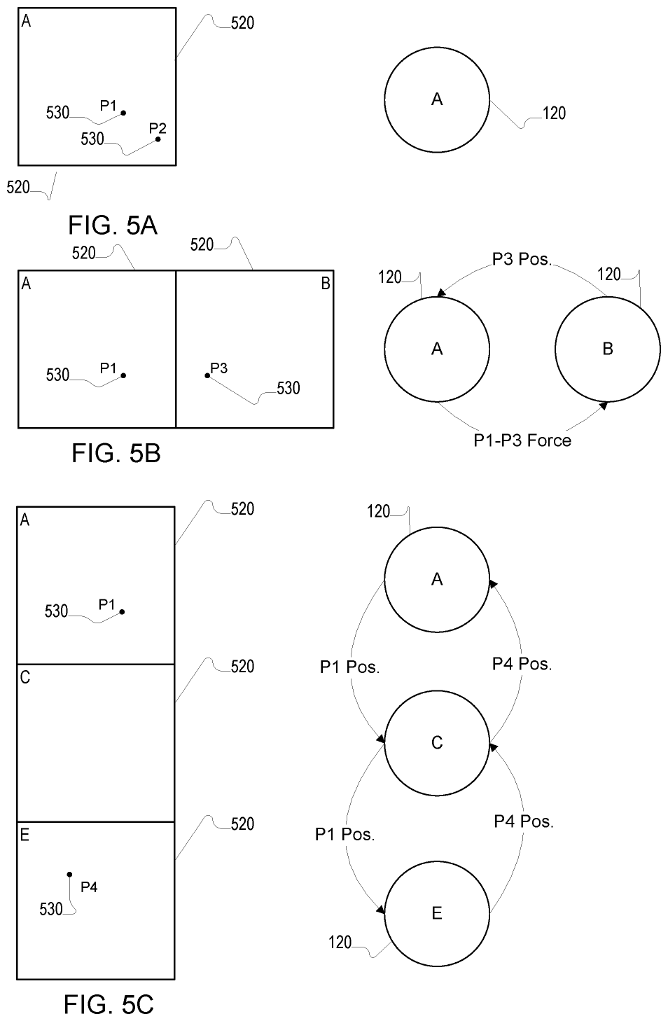

Referring to FIGS. 5A-C, there are at least three ways that an interaction between two atoms may be computed in the system. Referring to FIG. 5A, if two atoms P1 and P2 (530) are in the same homebox A (520), then the interaction between atoms P1 and P2 may be computed at the homebox's node A (120) yielding a force term for computing the total force on P1 as well as the force term for computing the total force on P2 resulting from interaction with P1 (e.g., an equal and opposite force). No inter-node communication is needed for computing these terms because the node already has the data for both atoms.

Referring to FIG. 5B, one way to compute the interaction between two atoms that are in different home boxes, for example, atoms P1 and P3 (530) in homeboxes (520) A and B, respectively, the position information for P3 is communicated from node B to node A. Once node A has the information for both atoms P1 and P3, it can compute the interaction between the two atoms. Node A keeps the force exerted on P1 by P3 for accumulating into the total force on P1, and sends the force exerted on P3 by P1 (denoted the “P1-P3” force) from node A to node B. At node B, the P1-P3 force is accumulated into the total force on P3. Note that only one node computes the interaction between P1 and P3, and node A does not need to send the position information for P1 to node B (at least for the purpose of computing the P1-P3 interaction).

Referring to FIG. 5C, another way to compute the interaction between two atoms that are in different home boxes, for example, atoms P1 and P4 (530) in homeboxes A and E, respectively, the position information for P1 is communicated from node A to node E and the position information for P4 is communicated from node E to node A. Node A computes the P4-P1 interaction, and node E also computes the P1-P4 interaction. Node A uses the result to accumulate into the total force on P1 and node E uses its result to accumulate into the total force on P4. There is no need for node A to send the P1-P4 force term to node E, nor is there a need for node E to send the P4-P1 force to node A. Note that homeboxes A and E are not necessarily adjacent and therefore communication between nodes A and E are indirect, for example as illustrated, via another node C.

As shown above, for example, with reference to FIGS. 5B and 5C, one approach to computing the pairwise interactions between atoms that are within a cutoff radius of each other but not located in a same homebox is to import the data for all the atoms within the cutoff radius of a homebox to that node's homebox. Note that the determination of which atom to import (or conversely which atoms to export from the nodes of their homeboxes) can be based on specification of the region from which atoms must be imported. This region may be defined in a conservative (i.e., worst case) manner such that the import region is guaranteed to import all atoms regardless of the specific location of an atom in the importing node's homebox or the specific location of an atom that is imported in the import region. Therefore, the import region for a node can be based on the cut-off radius and the geometric volumes of the homebox and the nearby homeboxes, and is in general determined prior to the start of the simulation without consideration of the specific locations of atoms in the simulation volume. The import region used in this example can be referred to as a “full shell” import region.

In this example, while two atoms that are already in a node's homebox have their interaction computed at that node as illustrated in FIG. 5A, the node applies a hybrid approach to determining whether it uses the approach illustrated in FIG. 5C to compute an interaction between atoms from different homeboxes, or otherwise to use the approach in FIG. 5B. For interactions that use the approach in FIG. 5B, while the nodes of the homeboxes of each of the atoms have sufficient information to compute the interaction, both nodes use an identical rule to determine which of the nodes is to compute the interaction.

One example of the rule to determine which of the two nodes for a particular pair of atoms is to compute the interaction is referred to below as a “Manhattan distance” rule. This rule can be stated as follows. The interaction between the two atoms is computed on the node whose atom of the two has a larger Manhattan distance (the sum of the x, y, and z distance components) to the closest corner of the other node's homebox. In the example, illustrated in FIG. 5B, atom P1 has a larger Manhattan distance to the closest corner of homebox B than the Manhattan distance of atom P2 to the closest corner of homebox A, and therefore node A computes the interaction between P1 and P2, and node B does not compute the interaction (or at least does not double count the result of such a computation if for some reason it computes it). Note that the Manhattan distance rule is just one computationally efficient distributed rule for making the selection, for example, between nodes A and B in FIG. 5C, yet it should be recognized that yet other rules can be used.

The decision of whether to use the approach illustrated in FIG. 5C, in which the computation of interaction between two atoms is computed at two nodes, or whether to use the approach illustrated in FIG. 5B, in which the computation is performed at one node, and the result returned to the other node, is generally based on latency considerations. For example, while computing the interaction at only one node may reduce the total amount of computation, it introduces the communication cost of returning the result of the computation to another node. This cost affects total inter-node network traffic, but perhaps more importantly introduces latency. Such latency may be significant if there are multiple “hops” in the path between the two nodes.

One approach to a node making the decision of whether to apply the Manhattan distance rule (FIG. 5B) or to apply the approach illustrated in FIG. 5C (which may be referred as the “full shell” rule) is based on the network proximity between the nodes. For a particular node, the nodes that provide the atoms in the import region for the node are divided into close and far neighbors. In one example, close neighbors to a node are those nodes that have direct inter-node connections (e.g., links 110 in FIG. 1), while far neighbors have indirect (i.e., multiple hop connections (e.g., over multiple links 110). An example of a proximity-based decision is to apply the Manhattan distance rule to all atoms imported from near neighbors, and to apply the full shell rule to atoms imported from far neighbors. The decision of near and far neighbors may be made differently, for example, to yield different tradeoffs between computation, network traffic, and communication latency, for example, defining near neighbors to be within one hop of each other and far neighbors if they are two or more hops from each other. Also, in at least some examples, all the neighboring nodes made be determined to be near or to be far. Although in general, the definition of near and far is the same for all nodes, it may be possible to have a different definition at different nodes, for example, based on considerations such as the expected number of atoms in proximity to the node.

Therefore, as an example, the procedure that is applied at a node in the hybrid approach is as follows:
(a) if two atoms are located in the same homebox, the interaction between those two atoms is computed at the node of that homebox, and that computation yields the respective forces that are aggregated into the total force for each of the two atoms;
(b) if two atoms are in different homeboxes, and there is a direct communication link between the nodes of those homeboxes, the interaction between the two atoms is computed on the node whose atom of the two has a larger Manhattan distance (the sum of the x, y, and z distance components) to the closest corner of the other node's homebox, and the data (e.g., the position data) for the atom at the nodes where the computation is not computed is sent from the node maintaining the data for that node, and the force on that atom is returned back to the node where it will be aggregated;
(c) if two atoms are in different homeboxes that are not directly linked, then the interaction is computed in each of the two homeboxes by exchanging the data for each atom at the start of the time step, but not requiring return of the computed forces due to the redundant computation of the same result at both nodes.

3 Specialized Pairwise Interaction Modules

As introduced above, a given node 120 receives data for atoms from nearby nodes in order that it has all the needed data for all for the pairwise interactions assigned to it for computation, for example according to the hybrid rule introduced above. Also as introduced above, by virtue of the import region for a node being defined conservatively, in general there are at least some pairs of atoms available for computation at a node whether the two atoms are separated by more than the cutoff radius.

Generally, for each of the atoms in the homebox of the node, the node excludes any pairwise computation with other atoms (i.e., with imported atoms) that are beyond the cutoff radius. For a pair of atoms that are within the cutoff radius, the node determines whether the computation is assigned to that node, for example, according to the hybrid rule described above.

During each simulation time step, in an intra-node communication process described further below, data for a first set of atoms is stored in the PPIMs 132 of the node with each atom of the first set being stores at a subset (generally less than all) of the PPIMs. Then data for a second set of atoms is streamed to the PPIMs. The communication process guarantees that each pair of potentially interacting atoms, with one atom from the first set and one atom from the second set, is considered for computation at exactly one PPIM. In some examples, the first set of atoms consists of the atoms in the node's homebox, and the second set of atoms consists of the atoms in the node's homebox as well as the imported atoms from the import region. More generally, the decision of what constitutes the first set and the second set is such that all pairs of interactions between an atom in the first set and an atom in the second set is considered at exactly one PPIM of the node.

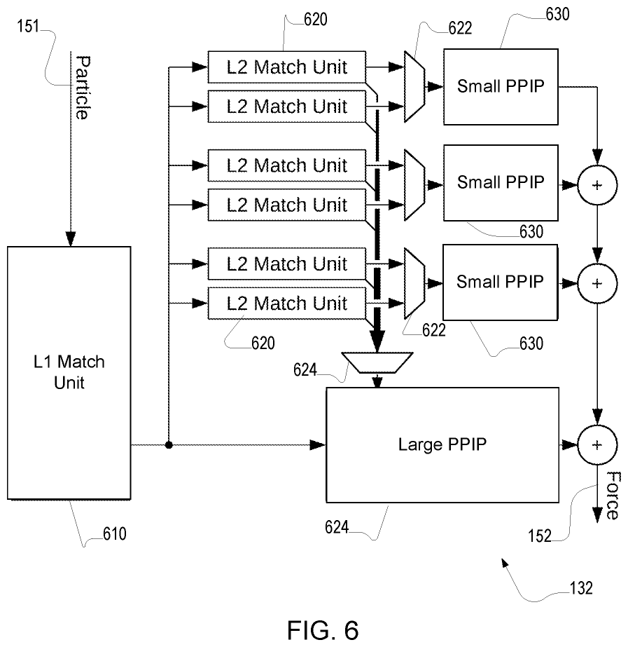

Referring to FIG. 6, the atoms of the first set of atoms that are assigned to a particular PPIM 132 (i.e., the PPIM illustrated in FIG. 6, which is one of many PPIMs) are stored in (or otherwise available from a memory coupled to) a match unit 610. In some embodiments, the match unit 610 is implemented as a parallel arrangement of a number of separate match units, for instance 96 such units. The implementation of one or parallel match units together is to receiving the data for an atom of the second set and to form matched pairs of that atom and atoms of the first set for further consideration while excluding from further consideration such pairs that are guaranteed to be farther apart than the cutoff radius. In general, at least some of the matched pairs of atoms are separated by more than the cutoff radius. Match unit 610 is referred to as a “level 1 (L1)” match unit because it makes a conservative decision by matching the arriving atom of the second set with each stored atom of the first set according to a computation that requires fewer operations than an exact computation of separation. One example of such a reduced operation computation is a determination of whether the second atom is within a polyhedron centered at the location of the atom of the first set. The polyhedron is selected to completely contain a sphere of the cutoff radius (i.e., it is guaranteed not to exclude any pairs of atoms at or closer to each other than the cutoff radius), that therefore no pair of atoms is improperly excluded, but there are in general some excess pairs that are matched. The computation of whether the atom of the second set is within the polyhedron requires less computation than the summing to the squared distances between the atoms in the three dimensions required for accurately computing the true distance between the atoms. One example of a polyhedron is defined by the inequalities: |Δx|+|Δy|+|Δz|≤\sqrt{3}R_{cut}, |Δx|≤R_{cut}, |Δy|≤R_{cut}, and |Δz|≤R_{cut}. Note that checking of these inequalities does not require any multiplications and optionally may use lower-precision arithmetic and comparison circuitry, and furthermore other low-complexity matching calculations may be used (e.g., adding further inequalities to create a small polyhedron volume).

Each of the pairs of atoms retained by the match unit 610 (i.e., because it passes all the inequalities defining the polyhedron) is passed to one of a set of match units 620, which are referred to as “level 2 (L2)” match units. The particular L2 match unit to which a pair is passed is selected based on a load balancing approach (e.g., round robin). In this example, each of these L2 match units makes a three-way determination by first computing a high-precision inter-atom distance or squared distance (e.g., d=(Δx)²+(Δy)²+(Δz)²) and then either (a) determining that the computed distance is greater than the cutoff radius (i.e., the L1 match unit 610 found the pair to match based on the approximate bounding polyhedron), (b) determining that the pair of atoms are separated by a distance between a mid-distance and the cutoff radius, and (c) determining that the atoms are separated by less than the mid distance. In some examples, the cutoff radius may be 8 Angstrom, while the mid distance may be 5 Angstrom.

If the distance between the atoms of a pair is determined to be greater than the cutoff radius, the pair of atoms is discarded by the L2 match unit 620. If the distance is determined to be between the mid distance and the cutoff radius, the pair is passed from the L2 match unit via a multiplexor 622 to a “small” Particle-Particle Interaction Pipeline (PPIP) 630. If the distance is determined to be less than the mid distance, the pair is passed from the L2 match unit via a multiplexor 624 to a “large” PPIP 624. As the PPIPs 630, 624 compute the force terms on the atoms, these forces are passed out from the PPIM.

There may be one or more differences between a “small” PPIP 630 and a “large” PPIP 624. One difference that may be exploited is that because the distance between atoms of pairs processed by a small PPIP 630 is at least the mid distance, the magnitude of the force is in general smaller than when then the atoms are closer together. Therefore, the hardware arithmetic units of the small PPIP can use fewer bits by not having to accommodate results beyond a certain magnitude, which can result in fewer logic gates. For example, multipliers scale as the square of the number of bits (w²) and adders scale super-linearly (w log w). For example, the large PPIP may gave 23-bit data paths while the small PPIPs may have 14-bit data paths. In some embodiments, other reductions in hardware complexity may be used, for example, by simplifying the form of the force calculation or by reducing the precision (e.g., removing least significant bits) of the representation of the resulting forces.

Conversely, the large PPIP 624 accommodates computation of interactions between nearby atoms, which may require more bits to represent the potential magnitude of the force between nearly atoms. In some embodiments, the form of the force calculation may be more complex and computationally intensive, for example, to provide accuracy even when atoms are very close together.

The selection of the mid-radius may be based on various considerations, for a load balancing consideration to distribute load between the large versus the small PPIPs, or based on the computational capabilities of the PPIPs. Based on the volumes of the sphere of the cutoff radius and the sphere of the mid radius, with a ratio of 8:5 the number of interactions expected to be considered by the small PPIPs vs the large PPIP is approximately 3:1, which motivates implementing three small PPIPs 630 and one large PPIP 624 per PPIM 132. In a hardware implementation, the three small PPIPs consume approximately the same circuit area and/or the same power as the one large PPIP.

In some alternatives, the decision of whether to route a matched pair of atoms to the large PPIP versus a small PPIP may be based in addition or instead on the nature of the interaction between the two atoms. For example, the L2 match unit may determine that based on characteristics of the pair of atoms, that the large PPIP is required even though the separation is more than the mid radius.

Regardless of the path taken within the PPIM (i.e., via a small PPIP or the large PPIP) for an atom of the second set that arrives at the PPIM is the particle bus 151, the results of the force computations are emitted via the force bus 152 from the PPIM.

4 Particle Interaction Table

As introduced above, atoms have changing (i.e., “dynamic”) information associated with them such as their position and their velocity, which are updated at simulation time steps based on forces applied to them from different atoms. Atoms also have static information that does not change during the simulation period. Rather than passing this static information between nodes along with the dynamic information for a node, the data for atom passing between nodes includes metadata, for example, a unique identifier and an atom type (referred to as its “atype”) that accompanies the dynamic information that is transmitted. The atype field can be used, for example, to look up the charge of an atom in the PPIM. Different atypes can be used for the same atom species based of its covalent bond(s) in a molecule.

After matching two atoms, for example, in the L1 match unit 610, and prior to computing an interaction between two atoms, the type of interaction is determined using an indirect table lookup. For example, the L1 match unit, or alternatively an L2 match unit, determines the atype for each atom, and separately for each atom determines an extended identifier for that atom, for example, based on a table lookup. The pair of extended identifiers are then together as part of an index that is used to access an associative memory (e.g., existing within or accessible to the L1 or L2 match unit) to yield an index record that determines how the computation of the interaction between the two atoms is to be computed. For example, one of an enumerated set of computation functions (e.g., a functional form) may be identified in a field of the index record. The identifier of the functional form may then accompany the metadata for the two atoms as they pass to a large or a small PPIP. In some examples, the functional form may also determine to what type of PPIP the matched pair is to be routed to, for example, if some functional forms can be computed by the large PPIP and not by the small PPIP.

5 Communication Compression

As discussed above, at each simulation time step, nodes export atom position information to nearby nodes in their export region such that all the nodes receive all the atoms in their respective import regions. Note that in the examples described above, the export region of a node is the same as the import region of the node.

In general, atom positions in simulations change slowly and smoothly over time, offering an opportunity for data compression. Therefore, when a node sends atom position information at successive simulation time steps, the positions will in general have changed very little from time step to time step. Various forms of compression of the amount of data (i.e., the number of bits that need to be communicated) reduce the communication requirements, thereby reducing the time needed to transfer the atom information between nodes.

One approach to compression is enabled by a receiving node maintaining a cache of the previous position (or more generally history of multiple previous positions) of some or all of the atoms that it has received from nodes in its import region. The sending node knows for which atoms the receiving node is guaranteed to have cache information, and the cache information at the receiving node is known exactly to both the receiving node and the sending node. Therefore, when a node A is sending (i.e., “exporting”) position information for an atom to node B, if node A knows that node B does not have cached information for that atom (or at least is not certain that it does have such cached information), node A sends complete information. When node B receives the position information for that atom, it caches the position information for use at a subsequent simulation time step. If on the other hand node A knows that node B has cache information for the atom, compressed information that is a function of the new position and the cached information for that atom may be sent from node B to node A. For example, rather than sending the current position, node A may compute a difference between the previous position and the current position and send the difference. The receiving node B receives the difference, and adds the difference to the previous position to yield the new position that is used at the receiving node B. As discussed further below, in general, the difference has a substantially smaller magnitude than the absolute position within node B's homebox, and therefore fewer bits (on average) may be needed to communicate the difference. Other compressions may be possible, for example, more than simply the cached prior position for an atom. For example, with two prior positions for an atom, the nodes can approximate a velocity of the atom, make a prediction from the prior positions, and then compute a difference from the prediction and the actual position. Such a prediction may be considered to be a linear prediction (extrapolation) of the atom's positions. This difference may, in general, have on average an even smaller magnitude than the difference from the prior position. Various alternative prediction functions may be used, as long as both the sending node and the receiving node use the same prediction function, and both nodes have the same record of prior positions (or other summary/state inferred from the prior positions) from which to make the prediction. For example, with three prior positions, a quadratic extrapolation of the atom's position may be used.

There are a number of alternative ways that a sending node may know which atoms are cached at a receiving node. One way is to provide ample memory at each node so that if a node sends position information at one time step, it can be assured that the receiving node has cached information for that node at the next time step. Another way is for both the sending node and the receiving node to make caching and cache ejection decisions in identical ways, for example, with each node having fixed numbers of cache locations for each other's node, and a rule for which atoms to eject or not cache when the locations are not sufficient to cache all the atoms. Yet another way is for the node to send explicit information back to the sending node regarding whether the atom is cached or not, for example, in conjunction with force information that may be sent back to the atom's homebox node. In yet other alternatives, there may be a preference to cache atoms that require more “hops” via the inter-node network, thereby reducing overall network usage.

There are a number of alternative circuit locations where to maintain the cache information at a node. In one alternative, the cache information is maintained at the edge of the node, for example, in the edge network tiles 122 (see, e.g., FIG. 4). For example, the cache information may be maintained at the channel adapters 115. In examples in which a particular atom may arrive over a different link 110 at different time steps (e.g., due to routing differences from time step to time step), the cache information may be accessible to multiple channel adapters 115 either through a shared memory or by replication to the channel adapters. In some alternatives, the cache information may be stored and applied elsewhere, for example, in a match unit of a PPIM.

Having reduced the magnitude of the position information that is sent from node to node, one way to take advantage of the smaller magnitude is to use a variable length encoding of the information. For example, leading zeros of the magnitude may be suppressed or run-length encoded (e.g., by using a magnitude followed by sign bit encoding, such that small negative and small positive quantities have leading zeros). The number of leading zeros may be represented by an indicator of the number of leading zero bytes, followed by any non-zero bytes. In some examples, multiple differences for different atoms are bit-interleaved and the process of encoding the length of the leading zero portion is applied to the interleaved representation. Because the differences may tend to have similar magnitudes, the length of the leading zero portion may be more efficiently encoded using the interleaved representation.

In experimental evaluation of this compression technique, approximately one half the communication capacity was required as compared to sending the full position information. To the extent that communication delays contribute to the time required to simulate each time step, such reduction in the total amount of communication reduces the total amount of real time needed to simulate a given simulation duration. Furthermore, in some experimental evaluations, the reduction in communication requirements removes communication from being a limiting factor on simulation speed.

6 Network Fences

As may be appreciated, the distributed computation at the nodes of the system requires a degree of synchronization. For example, it is important that when performing computations at a node for a particular simulation time step, the inputs (i.e., the atom positions) are those associated with the start of the time step and the results are applied correctly to update the positions of atoms at the end of that time step. One approach to synchronization makes use of hardware synchronization functions (“primitives”) that are built into the inter-node network. One such primitive described below is referred to as a “network fence.”

Network fences are implemented with fence packets. The receipt at a node B of a fence packet send from node A notifies node B that all packets sent from node A before that fence packet from node A have arrived at node B. Fence packets are treated much like other packets sent between nodes of the system. Network features including packet merging and multicast support reduce the total communication requirements (e.g., “bandwidth”) required to send the fence packets.

Each source component sends a fence packet after sending the packets it wants to arrive at destinations ahead of that fence packet. The network fence then guarantees that the destination components will receive that fence packet only after they receive all packets sent from all source components prior to that fence packet. The ordering guarantees for the network fence build on an underlying ordering property that packets sent along a given path (e.g., in a particular dimensional routing order) from source to destination are always delivered in the order in which they were sent, and the fact that a fence packet from a particular source is multicast along all possible paths a packet from that source could take to all possible destinations for that network fence.

Addressing in the network permits packets to be addressed to specific modules or group of modules within a node. For example, a packet may be addressed to the geometry cores 134 of a node while another packet may be addressed to the ICM modules 150 of the node. In some examples, the fence packet transmitted from a node includes a source-destination pattern, such as geometry-core-to-geometry-core (GC-to-GC) or geometry-core-to-ICB (GC-to-ICB), and a number of hops. The function of the fence is then specific to packets that match the pattern. The number of hops indicates how far through the network the fence message is propagated. For example, the receipt of a GC-to-ICB pattern fence packet by an ICB indicates it has received all the atom position packets sent prior to this fence packet, from all GCs within the specified number of inter-node (i.e., torus) hops. This is a common use-case in simulation the import region from which a node receives atom information has a maximum number of inter-node hop from any source node in the import region. By limiting the number of network hops, a network fence can achieve reduced latency for a limited synchronization domain. Note that each source within the maximum number of hops sends the fence packet, and therefore a receiving node know how many fence packets it expects to receive based on the fixed interconnection of nodes in the network.

To limit the communication requirements of propagating fence packets, examples of the inter-node network implement merging and/or multicast functions described below.

When a fence packet arrives at an input port of a node (i.e., arriving at an edge router 143 of the node), instead of forwarding the packet to an output port, the node merges the fence packet. This merging is implemented by incrementing a fence counter. When the fence counter reaches the expected value, a single fence packet is transmitted to each output port. In some examples, a fence output mask is used to determine the set of output ports that the fence should be multicast to. One way this determination is made, for input port i, bit j of its output mask is set if the fence packet needs to travel from the input port i to the output port j within that router. When the fence packet is sent out, the counter is reset to zero. Because the router can continue forwarding non-fence packets while it is waiting for the last arriving fence packet, normal traffic sent after the fence packet may reach the destination before the fence packet (i.e., the network fence works as a one-way barrier).

The expected count and the fence output mask are preconfigured by software for each fence pattern. For the example a particular input port of may expect fence packets from two different paths from upstream nodes. Because one fence packet will arrive from each path due to merging, the input port will receive a total of two fence packets, thereby setting the expected count to two. The fence counter width (number of bits) is limited by the number of router ports (e.g., 3 bits for a six-port router). The fence output mask in this example will have two bits set for the two output ports to which the fence packets are multicast.

The routing algorithm for the inter-node torus network exploits the path diversity from six possible dimension orders, as well as two physical channel slices for each connected neighbor. In addition, multiple virtual circuits (VCs) are employed to avoid network deadlock in the inter-node network, meaning that fence packets must be sent to all possible VCs along the valid routes that packets can travel. When the network fence crosses the channel, fence packets are thus injected to the edge network 144 by the channel adapter 115 on all possible request-class VCs. Although some hops may not necessarily utilize all of these VCs, this rule ensures that the network fence covers all possible paths throughout the entire network and simplifies the fence implementation because an identical set of VCs can be used regardless of the number of hops the packet has taken. Within the edge router 143, a separate fence counter must be used for each VC; only the fence packets from the same VC can be merged.

The description above is limited to a single network fence in the network. By adding more fence counters in routers, the network supports concurrent outstanding network fences, allowing software to overlap multiple fence operations (e.g., up to 14). To reduce the size requirement for the fence counter arrays in the edge router, the network adapters implement flow-control mechanisms, which control the number of concurrent network fences in the edge network by limiting the injection of new network fences. These flow-control mechanisms allow the network fence to be implemented using only 96 fence counters per input port of the edge router.

A network fence with a geometry-core-to-geometry-core (GC-to-GC) pattern can be used as a barrier to synchronize all GCs within a given number of torus hops; once a GC has received a fence, then it knows that all other GCs have sent one. Note that when the number of inter-node hops for a GC-to-GC network fence is set to the machine diameter (i.e., the maximum number of hops on the 3D torus network to reach all nodes), it behaves as a global barrier.

7 Intra-Node Data Communication

At the beginning of a simulation time step each core tile 124 of a node has stored in its memory a subset of the atom positions computed during the previous time step for atoms that are in the homebox for that node. During the computation for the time step, these positions will be needed at PPIMs of that node, and will also be needed at nodes within the export region of that node. As introduced above, the node has a 2D mesh network with links 142 and core routers 141. The positions of the atoms are broadcast over columns of the 2D mesh network such that the PPIMs in each column have all the atoms stored in any core tile in that column at the start of the time step. Concurrently, each core tile sends the atom positions along rows of the 2D network to the edge tiles 122 on each edge in the same row of the node. The edge tiles are responsible for forwarding those atom positions to other nodes of the export region of the node.

Once all the PPIMs have copies of the atom positions, then any other atom that passes via a position bus 151 from one edge to the other is guaranteed to encounter each atom in the node's homebox in exactly one PPIM, and as described above, may be matched if the two atoms are within the cutoff radius of each other. Therefore, the computation of pairwise interactions between atoms in the PPIMs may be commenced in the node.

The initial PPIM computations requires only node-local atom information (i.e., interactions between atoms that are both in the node's homebox), with each core tile broadcasting its atom positions over the row of PPIMs over the position bus 151, thereby causing all the node-local computations (see, e.g., FIG. 5A) to be performed. The resulting force components are broadcast over the force bus 152, and are retrieved at the core times where the atom's information is stored.

As atom position information arrives at the edge tiles from other nodes, the position information streamed from the edge tiles across the rows of core tiles from the edge tiles, thereby causing each atom that is imported to encounter each atom in the node's homebox to be encountered at exactly one PPIM. These interactions produce forces that are accumulated in the PPIM for atoms for which the node is responsible for computing and accumulating the forces and/or streams the forces back over the force bus 152 to the edge tile for return to the node that provided the position information for the interaction. When the streaming is complete, the accumulated forces in the PPIMs are communicated in columns of the core tile and passed to the core tiles that hold the atoms' information.

After the core tile has received all the force terms, whether from other core tiles on the same node, or returned from other nodes, the core tile can use the total forces to perform the numerical integration, which updates the position of each atom.

In the implementation described above, because each column has 12 core tiles and 2 PPIMs per core tile for a total of 24 PPIMs per column, there is a 24× replication of the atom information for a node's homebox. While this replication is effective to provide parallel computation, alternatives do not require this degree of replication. For example, while the full 24× replication permits any atom to be matched to be passed over a single position bus 151 and be guaranteed to encounter all atoms in the node's homebox, less replication is possible by passing each atom over multiple position busses. For example, with no replication over core tiles of a column and dividing the atoms of a core tile among the two PPIMs of the core tile, each atom may be sent over all position busses 151 and be guaranteed to encounter each homebox atom in exactly one PPIM. Intermediate levels of replication may also be used, for example, with core tiles may be divided into subsets, and each atom is then required to be sent over one position bus of each subset to encounter all the homebox atoms.

As yet another alternative implementation, a paging approach to access of the atoms of a homebox may be used. In such an approach, the ICB 150 may load and unload stored sets of atoms (e.g., using “pages” of distinct memory regions) to the PPIMs, and then each atom may be streamed across the PPIMs once for each set. Therefore, after having been streamed multiple times, the atom is guaranteed to have encountered each of the node's homebox atoms exactly once. At the end of each “page”, the PPIMs stream out the accumulated forces on their homebox atoms.

8 Bond Calculation

As introduced above with reference to FIG. 3, each core tile includes a bond calculation module (BC) 133, which is used by the tile for interactions between atoms that are directly, or in some configurations indirectly, bonded. Not all bonded forces are computed by the BC. Rather, only the most common and numerically “well-behaved” interactions are computed in the BC, while other more complex bonded calculations are computed in the geometry cores 134. Note that this is somewhat analogous to using the small PPIPs for computing a subset of interactions, and using the large PPIP to compute the remaining interactions, which may require more complex interaction formulations.

The BC determined forces, including stretch, angle, and torsion forces. For each type of bond, the force is computed as a function of a scalar internal coordinate (e.g., a bond length or angle) computed from the positions of the atoms participating in the bond. A GC 134 of the tile (i.e., one of the two GCs of the tile) sends these atom positions to the BC 133 to be saved in a small cache, since an atom may participate in multiple bond terms. Subsequently, the GC sends the BC commands specifying the bond terms to compute, upon which the BC retrieves the corresponding atom positions from the cache and calculates the appropriate internal coordinate and thus, the bond force. The resulting forces on each of the bond's atoms are accumulated in the BC's local cache and sent back to memory only once per atom, when computation of all that atom's bond terms is complete.

9 Exponential Differences In some examples, interactions between particles take the form of a difference of exponentials, for example, of the form exp(-ax)-exp(-bx), or as the evaluation of an integral representing a convolution of electron cloud distributions. While it may be possible to compute the two exponentials separately and then take the difference, such differences may be numerically inaccurate (e.g., differences of very large numbers). A preferable approach is to form one series representation of this difference. For example, the series may be a Taylor series or a Gauss-Jacobi quadrature-based series. Furthermore, the number of terms needed to maintain precision of the overall simulation will in general depend on the values of ax and bx. Therefore, in computing the pairwise terms (e.g., in the small or large PPIP), different particular pairs of atoms, different information retrieved in index records for the pair, or different criteria based on the difference (e.g., absolute difference, ratio, etc.) in the values of ax and bx, can determine how many series terms to retain. By reducing the number of terms (e.g., to a single term for many pairs of particles), for example, when the two values are close, the overall computation of all the pairwise interactions may be reduced substantially while maintaining overall accuracy, thereby providing a controllable tradeoff between accuracy and performance (computation speed and/or hardware requirements).

10 Distributed Randomization

In some examples, the same values (e.g., forces on atoms) are computed redundantly in different processors, for example, to avoid communication cost. For example, such redundant computation may occur in the “Full Shell” method (e.g., in interactions as illustrated in FIG. 5C). There are also situations in which systematically truncating or rounding results may be detrimental to the overall simulation, for example, by introducing bias over a series of iterations. For example, repeatedly rounding down may make an integration over time significantly too small.

One approach to avoiding accumulated bias resulting from rounding is successive time steps is to add a small zero-mean random number before rounding or truncating a value computed for a set of particles. Such an approach may be referred to as dithering. However, when performing redundant computations in different processors, there is no reason that pseudo-random numbers generated at the different processors will necessarily be the same, for example, because of difference in the order of random number generation even if original seeds are the same. With different random numbers, the rounded or truncated values may differ, that the simulation may not stay in total synchronization (e.g., synchronization at an exact bit representation) across processors.

A preferred approach is to use data-dependent random number generation, where exactly the same data is used at all nodes that compute a value for a set of particles. One way to generate a random value is to use coordinate differences between the particles involved in the computation as a random seed for generating the random value(s) to be added before rounding or truncation. In some embodiments, the low order bits of the absolute differences in each of the three geometric coordinate directions are retained and combined as an input to a hash function whose output is used as the random value or that is used as a random seed of a pseudo-random number generator that generates one or more random numbers. When there are multiple computations involving the set of particles, the same hash is used to generate different random numbers to add to the results of computations. For example, one random number if split into parts, or a random number generator is used to generate a sequence of random numbers from the same seed. Because the values of the coordinate distances are exactly the same at all the processors, the hash value will be the same, and therefore the random numbers will be the same. Distances between particles may be preferable to absolute locations because the distances are invariant to translation and toroidal wrapping while absolute locations may not be. Computing differences in coordinate directions does not incur rounding error and therefore may be preferable to Euclidean (scalar) distances.

11 Summary

A number of different techniques are described above, for example, described in different numbered sections above. Unless otherwise discussed, these techniques may be individually chosen for inclusion in particular examples of the innovative simulation system and computational approach and that no particular technique is essential unless evident from the description. Furthermore, these techniques may be used independently or in conjunction with related techniques that are described in the Applicant's prior patents identified above.

As introduced above, the detailed description above focusses on the technical problem of molecular simulation in which the particles whose movement is simulated are atoms, yet the techniques are equally applicable to other multi-body (“N-Body”) simulation problems, such as simulation of planets. Some technique described above are also applicable and solve technical problems beyond multi-body simulation. For example, the approaches of dividing a set of computations among modules with different precision and/or complexity capabilities (e.g., between small and large PPIMs, or between the BC and GC modules) is a circuit design technique that can provide circuit area and/or power savings in other special purpose applications. Network fences, which provide in-network primitives that enforce ordering and/or signify synchronization points in data communication is widely applicable outside the problem of multi-body simulation, for example in a wide range of distributed computation systems, and may provide reduced synchronization complexity at computation nodes as a result. The technique for using data-dependent randomization to provide exact synchronization of pseudo random values at different computation nodes is also applicable in a wide range of distributed computation systems in which such synchronization provides an algorithmic benefit.

It should be understood broadly that molecular simulation as described above may provide one step in overall technical problems such as drug discovery in which the simulation may be used for example, to determine predicted properties of molecules and the certain for the simulated molecules are physically synthesized and evaluated further. Therefore, after simulation, at least some molecules or molecular systems may be synthesized and/or physically evaluated as part of a practical application to identify physical molecules or molecular systems that have desired properties.

A number of embodiments of the invention have been described. Nevertheless, it is to be understood that the foregoing description is intended to illustrate and not to limit the scope of the invention, which is defined by the scope of the following claims. Accordingly, other embodiments are also within the scope of the following claims. For example, various modifications may be made without departing from the scope of the invention. Additionally, some of the steps described above may be order independent, and thus can be performed in an order different from that described.

1.-4. (canceled)

5. An apparatus for molecular simulation using integrated circuits that are connected by network links to a toroidal network having nodes, each of which is one of said integrated circuits, wherein each of said integrated circuits includes core tiles, which are configured to estimate forces between atoms in a chemical system, a mesh network that interconnects said core tiles, and edge tiles, which manage movement communication between core tiles using said mesh network and, said edge tiles being connected to said network link to manage communication with another integrated circuit, said apparatus comprising, in each of said core tiles, interaction circuitry that receives a stream of information representative of streaming atoms and stores information representative of stored atoms, said interaction circuitry comprising a first interaction module, a second interaction module, and a matching circuit, said first and second interaction modules differ in computational complexity with said first interaction module carrying out more complex computations than said second interaction module, said matching circuit being configured to compare an interatomic distance between a pair of atoms and to cause said force between said atoms to be estimated using said first interaction module when said interatomic distance is less than a threshold and to use said second interaction module otherwise.

6. The apparatus of claim 5, wherein said second interaction module is one of a plurality of identical second interaction modules.

7. The apparatus of claim 5, wherein said second interaction module is one of three second interaction modules, there being three of said second interaction modules for each first interaction module.

8. The apparatus of claim 5, wherein said second interaction module is one of a number of identical second interaction modules, the number having been determined by a rule that increases the number as the threshold decreases.

9. The apparatus of claim 5, wherein said first interaction module is configured to estimate said force based on both electrostatic and quantum effects and wherein said second interaction module is configured to ignore said quantum effects when estimating said force.

10. The apparatus of claim 5, wherein said first interaction module consumes more area on said integrated circuit than said second interaction module.

11. The apparatus of claim 5, wherein said first interaction module consumes more energy per interaction calculation than said second interaction module.

12. The apparatus of claim 5, wherein said matching circuit comprises a first stage and a second stage, both of which compare said threshold with interatomic distances between atoms in a pair of atoms, said second stage carrying out a more accurate determination of interatomic distance than said first stage, wherein, after having compared said threshold with interatomic distances between atoms in a set of pairs of atoms, said first stage forwards said pairs to said second stage, which then determines said interatomic distance more accurately than said first stage.

13. The apparatus of claim 5, wherein said matching circuit is configured to consume a first amount of energy by comparing said threshold with interatomic distances between atoms in a set of pairs of atoms, to divide said set into first and second subsets, to discard pairs in said first subset, and to forward said pairs in said second subset for carrying out a second comparison between said threshold and interatomic distances between atoms in said second subset, said second comparison consuming more energy than said first comparison.

14. The apparatus of claim 5, wherein each atom is typed with a type index that is based on properties of said atom, wherein said integrated circuit includes first and second regions that store first and second information, said first information associating an interaction index with said type index and said second region associating a force-estimation method with said interaction index.

15. The apparatus of claim 5, wherein each atom is typed with a type index that is based on properties of said atom, an area of semiconductor substrate on said integrated circuit having been reserved for storing a dual-stage table in which a first stage of said table associates an interaction index with said type index and a second stage of said table stores information associating said interaction index with one of a plurality of interaction types.

16. The apparatus of claim 5, wherein a portion of a substrate of said integrated circuit comprises a geometry core formed thereon, said geometry core being in communication with said interaction circuitry and configured to support an interaction between atoms that is unsupported by said interaction circuitry, said interaction circuitry being configured to delegate estimation of said interaction to said geometry core.

17. The apparatus of claim 5, wherein a portion of a substrate of said integrated circuit comprises a geometry core formed thereon, said geometry core being in communication with said interaction circuitry, wherein said interaction circuitry estimates forces between atoms in a pair of atoms more than once, as a result of which redundant forces act on said atoms, wherein said geometry core is configured to subtract said redundant forces.

18.-22. (canceled)

23. An apparatus for molecular-dynamic simulation, said apparatus comprising an integrated circuit comprising tiles arranged in rows and columns, wherein each tile is disposed in a tile row and in a tile column, wherein each tile stores stored-set particles, receives streamed-set particles, and interacts said stored-set particles with said streamed-set particles, said streamed-set particles being streamed along said tile row, wherein each tile is configured to multicast its stored-set particles to other tiles in said tile column, whereby said stored-set particles are interacted with multiple streams of streamed-set particles at the same time.

24. A method for communicating data between processing nodes of a simulation apparatus, the communication including transmissions of data corresponding to a first body of a plurality of bodies being simulated, the transmissions including repeated transmissions of physical state information of said first body, said method comprising: storing first physical state data for said first body at a first processing node and at a second processing node; computing updated physical state data for said first body at said first processing node; computing predicted physical state data for said first body from said first physical state data at said first processing node and at said second processing node; determining a state data update from said predicted physical state date and said updated physical state data at said first processing node; transmitting said state update data from said first processing node to said second processing node; and determining said updated physical state at said second processing node from said first physical state data stored at said second node and said state update data received at the second processing node from the first processing node.

25. The method of claim 24, wherein said physical state data comprises a location of said first body.

26. The method of claim 25, wherein said physical state data comprises or can be used to compute a velocity of said first body.

27. The method of claim 25, wherein said predicted physical state data comprises a predicted location of said first body.

28. The method of claim 26, wherein said predicted physical state data comprises a predicted velocity of said first body.

29. The method of claim 24, wherein transmitting said state update data comprises transmission said data in a message that is smaller than a size required to send said updated physical state data.

30.-32. (canceled)
